(19) United States
(12) Patent Application Publication
(10) Pub. No.: US 2025/0083129 A1
(43) Pub. Date: Mar. 13, 2025

(54) PREPARATION METHOD OF MESOPOROUS CEO2-LOADED AU CATALYST, PRODUCTS THEREOF

(71) Applicant: Baotou Research Institute of Rare Earths, Baotou City (CN)

(72) Inventors: Cheng ZHANG, Baotou City (CN); Zhengping DONG, Baotou City (CN); Yan WANG, Baotou City (CN); Zhaoqiang LI, Baotou City (CN); Zhiyong DING, Baotou City (CN); Xin GUO, Baotou City (CN); Na KANG, Baotou City (CN); Yu WANG, Baotou City (CN); Yuanjiang WANG, Baotou City (CN); Liu YANG, Baotou City (CN); Hao QU, Baotou City (CN); Tiantian ZHANG, Baotou City (CN)

(21) Appl. No.: 18/675,793

(22) Filed: May 28, 2024

(30) Foreign Application Priority Data
Sep. 13, 2023 (CN) 202311177446.7

(51) Int. Cl. B01J 23/66; B01J 23/10; B01J 35/30; B01J 35/61; B01J 35/64; B01J 37/00; B01J 37/02; B01J 37/06; B01J 37/08; B01J 37/16; B01J 37/34; C07C 67/03

(52) U.S. Cl. CPC B01J 23/66; B01J 23/10; B01J 35/394; B01J 35/615; B01J 35/647; B01J 37/0018; B01J 37/0205; B01J 37/0236; B01J 37/06; B01J 37/08; B01J 37/16; B01J 37/343; C07C 67/03

(57) ABSTRACT

The present disclosure provides a preparation method of a mesoporous CeO₂-loaded Au catalyst, products and applications thereof, and belongs to the technical field of fine organic chemical industry. According to the present disclosure, firstly, COFs material COFs-41 is employed to prepare mesoporous CeO₂ carrier material with high specific surface area; then, Au nanoparticles loaded on the surface of mesoporous CeO₂ carrier pores are anchored by impregnation-reduction method to produce Au@CeO₂ catalyst with high specific surface area; and then, mesoporous Au@CeO₂ catalyst with high specific surface area, aromatic alcohols are added to a reactor, anhydrous methanol is used as a solvent, and heating is carried out to react in an atmosphere of atmospheric oxygen for a certain period of time at room temperature, and this catalyst catalyzes the oxidative transesterification of aromatic alcohols for synthesis of aromatic esters in a highly selective manner.

### CROSS-REFERENCE TO RELATED APPLICATIONS

This application claims priority to Chinese Patent Application No. 202311177446.7, filed on Sep. 13, 2023, the contents of which are hereby incorporated by reference.

### TECHNICAL FIELD

The present disclosure belongs to the technical field of fine organic chemical industry, and in particular to a preparation method of a mesoporous CeO₂-loaded Au catalyst, products and applications thereof.

### BACKGROUND

Esters are a class of compounds with a wide range of applications, usually used in the synthesis of various fine chemicals such as food and pharmaceuticals. Common methods for synthesizing esters include: reaction of organic acids with alcohols, synthesis of carboxylates with halogenated hydrocarbons, alcoholysis of nitriles, and esterification of carboxylic acid esters with alcohols. However, these traditional methods for synthesizing esters are accompanied by the generation of many environmentally polluting by-products, corrosion of manufacturing equipment, as well as the requirement of strict reaction conditions, and therefore, a proper method for synthesizing esters is required. From the economic and environmental point of view, direct one-pot oxidative esterification of alcohols or aldehydes is the most desirable method for the synthesis of esters.

To date, a large number of methods have been reported for the oxidative esterification of alcohols to esters. For example, homogeneous catalysts such as Au, Rh, and others are effective for catalytic oxidative esterification of alcohols. Cheng et al. successfully prepared esters in high yields by dehydrocoupling of aldehydes to alcohols as well as dehydrocoupling of tertiary alcohols with Rh-tripyridine catalysts (Chem. Sci., 2016, 7, 4428-4434). However, homogeneous catalysts usually require special ligands, higher reaction temperatures, and additives, which make the separation of products difficult. Therefore, in practical applications, non-homogeneous catalysts are usually used to replace homogeneous catalysts. In recent years, the preparation of non-homogeneous catalysts using transition metals is a popular topic in catalysis research. For example, a bifunctional Co-N co-doped carbon nonhomogeneous catalyst is obtained by calcining the chelates of citric acid, melamine, and CoC12-6H20 in an inert atmosphere using LiCl and KCl as templating agents, and the direct oxidative esterification of benzyl alcohol to methyl benzoate has a well-desired conversion (99.3%) and selectivity (99.4%) (Catal. Lett., 2019,149,3160-3168). Also, Singh et al. successfully synthesized 12-phosphotungstic acid anchored to MCM-41 as a bifunctional catalyst for the oxidative esterification of benzaldehyde with methanol. However, such catalysts usually involve high temperatures and pressures so as to enable the reaction to take place, which is highly energy-consuming and environmentally polluting, resulting in a very limited scope of application.

Numerous studies have shown that loaded Au non-homogeneous catalysts are effective in the direct oxidative esterification of benzyl alcohol and other aldehydes with methanol to obtain the corresponding esters. Numerous studies have shown that loaded Au non-homogeneous catalysts are effective in the direct oxidative esterification of benzyl alcohol and other aldehydes with methanol to obtain the corresponding esters. For instance, an iron-doped graphene-loaded Au catalyst for oxidative esterification of benzyl alcohol gives a conversion of 96.2% for benzyl alcohol and a selectivity of 99.9% for methyl benzoate. Moreover, Parreira et al. successfully loaded Au on hexagonal mesoporous silica containing Ce, Ti, and Fe modifications (Applied Catalysis A: General 397 (2011) 145-152). The synthesized non-homogeneous catalysts have been experimentally demonstrated to efficiently catalyze the oxidative esterification of benzyl alcohol and other aldehydes with methanol in one pot; however, a high temperature of 130 degrees Celsius (° C) is required to achieve a high conversion of benzyl alcohol. In addition, covalent organic frameworks (COFs), which are pre-designable crystals with highly ordered porous structures built on dynamic covalent chemistry and various organized building blocks, are widely used in the preparation of functional materials and organocatalysis. Xu et al. employed a non-stoichiometric strategy to introduce amino groups into the COF lattice to facilitate further encapsulation of Pd nanoparticles. The resulting non-homogeneous catalyst Pd/COF is used to catalyze the esterification of benzyl alcohol at 0.1 MPa O₂ and 90° C., with a conversion of 97%. Nevertheless, there is so far still no report on the preparation of complex catalysts using mesoporous CeO₂ with high specific surface area as a carrier, which in turn is used to catalyze the oxidative esterification of aromatic alcohols under relatively mild conditions (30-60° C.).

### SUMMARY

Aiming at the problems existing in the prior art, the present disclosure provides a preparation method, products and applications of a mesoporous CeO₂-loaded Au catalyst.

In order to achieve the above objectives, the present disclosure provides the following technical scheme.

A preparation method of a mesoporous CeO₂-loaded Au catalyst, where the preparation method includes: using covalent organic frameworks 41 (COFs-41) as a template and cerium nitrate as a cerium source, firstly loading Ce³⁺ by impregnation method, then preparing a mesoporous CeO₂ carrier material with high specific surface area by oxidative roasting; then, taking HAuCl₄ as a gold source and NaBH₄ as a reducing agent, loading Au nanoparticles on the mesoporous CeO₂ carrier material by impregnation-reduction method in an ethanol solution reaction system to obtain the mesoporous CeO₂-loaded Au catalyst.

The preparation method of the mesoporous CeO₂-loaded Au catalyst specifically includes following steps:
1) self-assembling raw materials 2,4,6-trimethylpyridine, benzoic anhydride and terephthalaldehyde in a closed tube system at 180 degrees Celsius (C) to synthesize a COFs-41 template material; ultrasonically dispersing the obtained COFs-41 template material in an ethanol solution containing cerium nitrate hexahydrate, and slowly removing the ethanol solvent at 60° C. by a rotary evaporator to obtain a powder, followed by slowly heating to 600° C. and holding for 3 hours (h) to obtain a black solid, and roasting at 500° C. for 3 h in an air to obtain a light yellow solid as the mesoporous CeO2 carrier material; and
2) dispersing the mesoporous CeO₂ carrier material in a mixed solution of water and ethanol, dropwise adding a methanol solution containing HAuCl₄·4H₂O while stirring, stirring for 8 h, then continuously dropwise adding a NaBH₄ solution, stirring for 6 h, washing and drying a product obtained by suction filtration to obtain the mesoporous CeO₂-loaded Au catalyst, namely an Au@CeO₂ catalyst.

Optionally, a mass ratio of the 2,4,6-trimethylpyridine, benzoic anhydride and terephthalaldehyde is 5:9:8.

Optionally, a mass ratio of the COFs-41 template material to the cerium nitrate hexahydrate is 1:5.

Optionally, a volume ratio of the water to ethanol is 1:9, and a solid-liquid ratio of the mesoporous CeO₂ carrier material to the mixed solution is 50 milligrams (mg): 1 milliliter (mL).

Optionally, a concentration of the HAuCl₄·4H₂O in the methanol solution containing HAuCl₄·4H₂O is 4.75 mg/mL.

The washing refers to washing solid powder samples with anhydrous ethanol and deionized water each for three times. The drying refers to vacuum drying at 80° C. for 3 h.

The present disclosure also provides an Au@CeO₂ catalyst prepared by the preparation method, where a loading amount of Au in the Au@CeO₂ catalyst is 1-3%, preferably 2%; a specific surface area is 153 square meter per gram (m²/g) and an average pore diameter is 8.7nanometers (nm).

The present disclosure also provide a method for catalyzing oxidative esterification of aromatic alcohols, whereby a catalytic oxidative esterification of aromatic alcohols is completed to synthesize aromatic esters compound by using aromatic alcohols as raw materials, using the Au@CeO₂ catalyst as a catalyst, and using anhydrous methanol as a solvent, and reacting with heating at room temperature and atmospheric pressure under an oxygen atmosphere. The reaction conversion and product yield are also analyzed by gas chromatography, taking benzyl alcohol as an example, and it is found that the conversion of benzyl alcohol is as high as 100%, and the selectivity of the product methyl benzoate is >99%. The reaction equation is as follows:

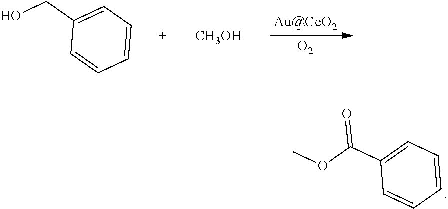

Optionally, a dosage ratio of the Au@CeO₂ catalyst, aromatic alcohols and anhydrous methanol is 10 mg: 1 millimole (mmol): 5 mL. A temperature of the heating is 30-60° C. and a duration is 5-48 h.

The aromatic alcohols include benzyl alcohol and benzyl alcohol derivatives. The Au@CeO₂ catalyst is recyclable in catalyzing the oxidative esterification of aromatic alcohols, with catalytic activity remaining after 10 cycles of use.

Compared with the prior art, the present disclosure has the following advantages and technical effects.

The mesoporous CeO₂ carrier material prepared by the present disclosure is obtained by using a COFs material with high specific surface area as a soft template, and the mesoporous Au@CeO₂ catalyst with high specific surface area is produced by the impregnation-reduction method, which is a simple preparation method involving mild conditions and good dispersion of active sites.

The Au@CeO₂ catalyst prepared by the present disclosure catalyzes the oxidative esterification of aromatic alcohol to synthesize aromatic carboxylic acid ester, with high conversion rate and good selectivity of aromatic carboxylic acid ester.

In the method for synthesizing aromatic carboxylic ester by oxidative esterification of aromatic alcohols provided by the present disclosure, the product and the catalyst are easy to be separated, the operation is simple, the method is economical and environment-friendly, and has a good application prospect.

### BRIEF DESCRIPTION OF THE DRAWINGS

The accompanying drawings, which constitute a part of this application, are used to provide a further understanding of this application. The illustrative embodiments of this application and their descriptions are used to explain this application, and do not constitute an improper limitation of this application. In the attached drawings:

FIG. 1A is a TEM image of Au(1%) @CeO₂ prepared in Embodiment 1 at 50 nm scale.

FIG. 1B is a TEM image of Au(1%) @CeO₂ prepared in Embodiment 1 at 20 nm scale.

FIG. 1C is a TEM image of Au(1%) @CeO₂ prepared in Embodiment 1 at 10 nm scale.

FIG. 1D is a TEM image of Au(1%) @CeO₂ prepared in Embodiment 1 at 5 nm scale.

FIG. 1E is a TEM image of Au(1%) @CeO₂ prepared in Embodiment 1 at 2 nm scale.

FIG. IF shows an HAADF-STEM plot of Au(1%) @CeO₂ prepared in Embodiment 1.

FIG. 2A shows the adsorption-desorption curve of CeO₂ carrier and Au/CeO₂ catalyst prepared in Embodiment 1 of the present disclosure.

FIG. 2B shows the pore diameter distribution of CeO₂ carrier and Au/CeO₂ catalyst prepared in Embodiment 1 of the present disclosure.

### DETAILED DESCRIPTION OF THE EMBODIMENTS

A number of exemplary embodiments of the present disclosure will now be described in detail, and this detailed description should not be considered as a limitation of the present disclosure, but should be understood as a more detailed description of certain aspects, characteristics and embodiments of the present disclosure.

It should be understood that the terminology described in the present disclosure is only for describing specific embodiments and is not used to limit the present disclosure. In addition, for the numerical range in the present disclosure, it should be understood that each intermediate value between the upper limit and the lower limit of the range is also specifically disclosed. The intermediate value within any stated value or stated range and every smaller range between any other stated value or intermediate value within the stated range are also included in the present disclosure. The upper and lower limits of these smaller ranges can be independently included or excluded from the range.

Unless otherwise specified, all technical and scientific terms used herein have the same meaning as commonly understood by one of ordinary skill in the art to which this present disclosure relates. Although the present disclosure only describes the preferred methods and materials, any methods and materials similar or equivalent to those described herein can also be used in the practice or testing of the present disclosure. All documents mentioned in this specification are incorporated by reference to disclose and describe methods and/or materials related to the documents. In case of conflict with any incorporated document, the contents of this specification shall prevail.

It is obvious to those skilled in the art that many improvements and changes can be made to the specific embodiments of the present disclosure without departing from the scope or spirit of the present disclosure. Other embodiments will be apparent to the skilled person from the description of the present disclosure. The specification and example of this application are only exemplary.

The terms “including”, “comprising”, “having” and “containing” used in this description are all open terms, which means including but not limited to.

The “room temperature” mentioned in the present disclosure is defined as 25±2° C. unless otherwise specified, and the “atmospheric pressure” mentioned is defined as 0.1013 MPa unless otherwise specified.

The raw materials used in the following embodiments of the present disclosure are all commercially available.

According to the preset disclosure, firstly, COFs material COFs-41 is employed to prepare mesoporous CeO₂ carrier material with high specific surface area; then, Au nanoparticles loaded on the surface of mesoporous CeO₂ carrier pores are anchored by impregnation-reduction method to produce Au@CeO₂ catalyst with high specific surface area; and then, mesoporous Au@CeO₂ catalyst with high specific surface area, aromatic alcohols are added to a reactor, anhydrous methanol is used as a solvent, and heating is carried out to react in an atmosphere of atmospheric oxygen for a certain period of time at room temperature, and this catalyst catalyzes the oxidative transesterification of aromatic alcohols for synthesis of aromatic esters in a highly selective manner.

In the method of the present disclosure, the preparation method of mesoporous Au@CeO₂ catalyst with high specific surface area is simple in operation and mild in conditions, and ultrafine Au nanoparticles are highly dispersed as active sites; combined with the synergistic catalytic effect between the active site of Au and the carrier CeO₂, the reaction selectivity of catalytic oxidative esterification of aromatic alcohols is effectively improved. The mesoporous Au@CO₂ catalyst with high specific surface area is used for catalytic oxidative esterification of aromatic alcohol, with high product selectivity, easy separation between the product and the catalyst, and easy industrial transformation and application.

The following embodiments serve as a further explanation of the technical schemes of the present disclosure.

### Embodiment 1

Preparation of mesoporous CeO2-loaded Au catalyst

1) 24.24 grams (g) of 2,4,6-trimethylpyridine, 45.25 g of benzoic anhydride and 40.24g of terephthalaldehyde are put into a closed tube system to synthesize COFs-41 template material at 180 degrees Celsius (° C.) by self-assembling; the obtained COFs-41 template material is ultrasonically dispersed in 50 milliliters (mL) ethanol solution containing 2.17 g cerium nitrate hexahydrate, and the ethanol solvent is slowly removed at 60° C. with a rotary evaporator, and the obtained powder is heated to 600° C. at a heating rate of 5 degrees Celsius per minute (° C./min) and kept for 3 h to obtain a black solid; the black solid is roasted in the air at 500° C. for 3 hours (h) to obtain a light yellow solid, which is the mesoporous CeO2 carrier material;

2) 500 milligrams (mg) of mesoporous CeO2 carrier material is dispersed in 10 mL of mixed solution of water and ethanol (the volume ratio of water to ethanol is 1:9), while stirring, 11mL of methanol solution containing 4.75 mg/mL of HAuCl₄·4H₂O is dropped into the CeO₂ dispersion system, and the mixture is stirred for 8 h, then 10 mL of NaBH₄ solution is dropped continuously, and the stirring is further continued for 6 h; the solid product obtained by suction filtration is washed with anhydrous ethanol and deionized water respectively for three times, and then dried in vacuum at 80° C. for 3 h, thus obtaining the mesoporous CeO2-loaded Au catalyst with high specific surface area, namely Au(1%) @CeO₂ catalyst.

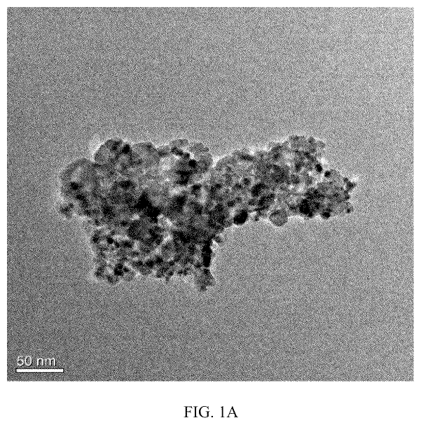

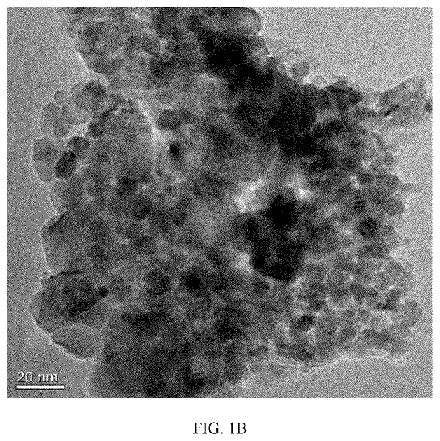

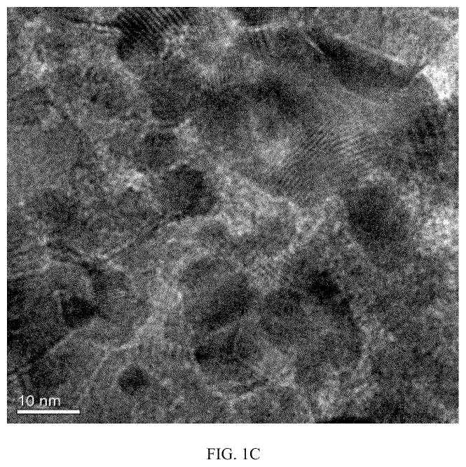

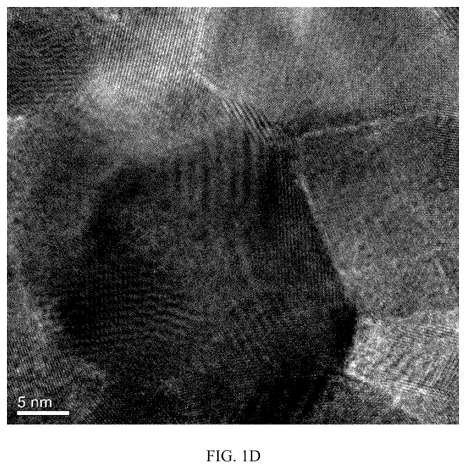

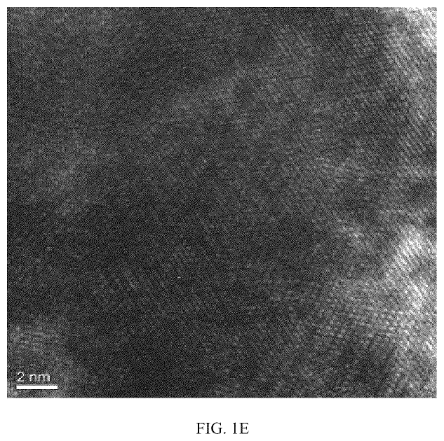

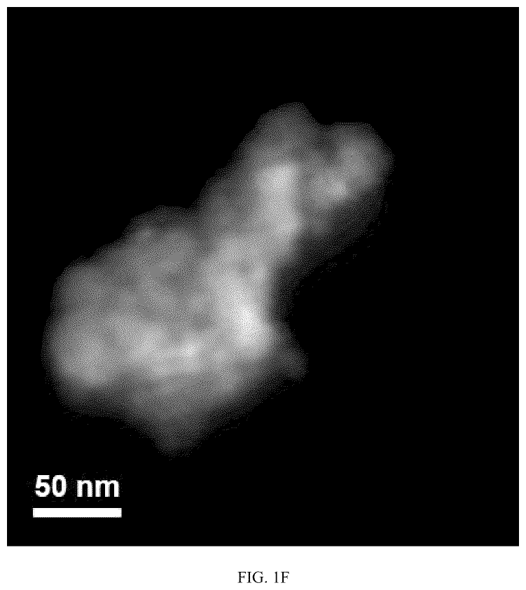

FIG. 1A-FIG. IF are the transmission electron microscope images and a Transmission electron microscopy (TEM) images of a CeO₂ carrier material prepared in Embodiment 1 of the present disclosure. From the figures, it is observed that Au(1%) @CeO₂ presents an amorphous structure, and no AuNPs is observed, indicating that Au is uniformly dispersed on the CeO² carrier.

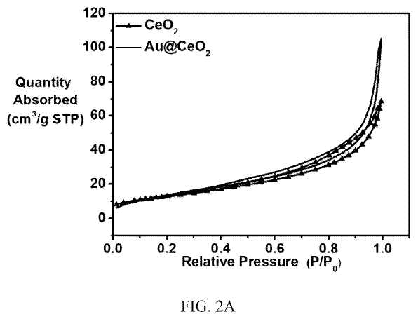

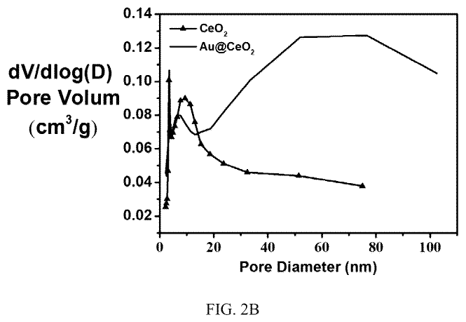

FIG. 2A shows the adsorption-desorption curve and FIG. 2B shows the pore diameter distribution diagram of CeO₂ and Au(1%)/CeO₂ catalyst prepared in Embodiment 1 of the present disclosure. As observed from the figures, both pure mesoporous CeO₂ and Au(1%) @CeO₂ show type IV isothermal curves, indicating that mesoporous structures exist in both materials, and the specific surface area of Au(1%) @CeO₂ is 115 square meter per gram (m²/g), and the pore diameters are 8.77 nm and 11.93 nm, respectively.

### Embodiment 2

The method of catalytic oxidative esterification of aromatic alcohols with Au@CeO₂ catalyst (product prepared in Embodiment 1) is as follows:
adding 10 mg of mesoporous Au@CeO₂ catalyst with high specific surface area, 1 millimole (mmol) of benzyl alcohol and 5 mL of anhydrous methanol into the reactor, reacting for 6 h at 50°° C. in atmospheric oxygen atmosphere, and selectively catalyzing oxidative esterification to synthesize methyl benzoate; the analysis by gas chromatograph shows that the conversion rate of the reaction is 100% and the yield of the product is 99%.

Embodiments 3-11

The Au(1%) @CeO₂ catalyst prepared in Embodiment 1 is used to catalyze the selective oxidative esterification of various benzyl alcohol derivatives, and the specific method is as follows.

10 mg of mesoporous Au(1%) @CeO₂ catalyst with high specific surface area and 1 mmol of benzyl alcohol derivative are added into the reactor, and then 5 mL of anhydrous methanol is added to react at room temperature and atmospheric oxygen atmosphere at 50° C. for a certain period of time to synthesize aromatic ester compounds by selective catalytic oxidative esterification. The reaction is monitored by gas chromatograph. See Table 1 for the specific reaction results.

TABLE 1
Results of selective oxidative esterification of benzyl alcohol catalyzed by Au(1%)@CeO₂ catalyst

| | | | Reaction | | |
|---|---|---|---|---|---|
| | | | duration | Conversion | Selectivity |
| Embodiments | Aromatic alcohols | Aromatic esters | (h) | rate (%) | (%) |
| Embodiment 3 | 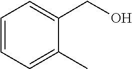 | 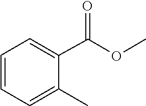 | 5 | >99 | 95 |
| Embodiment 4 | 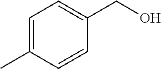 | 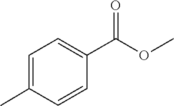 | 23 | 99 | 97 |
| Embodiment 5 | 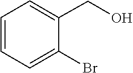 | 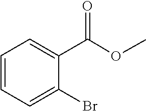 | 24 | 93 | 97 |
| Embodiment 6 | 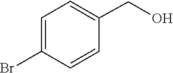 | 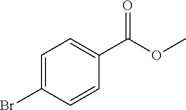 | 24 | 96 | 99 |
| Embodiment 7 | 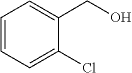 | 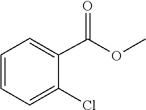 | 29 | 93 | 100 |
| Embodiment 8 | 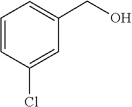 | 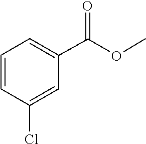 | 29 | 98 | 97 |
| Embodiment 9 | 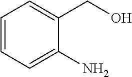 | 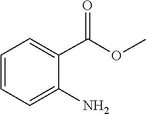 | 13 | 95 | 96 |
| Embodiment 10 | 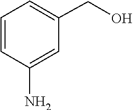 | 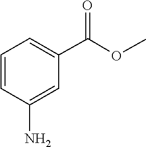 | 36 | 95 | 96 |
| Embodiment 11 | 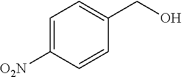 [SMILES: O=[N+]([O-])c1ccc(CO)cc1] | 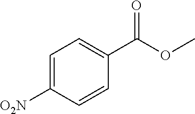 | 48 | 96 | 97 |

### Embodiment 12

The Au(1%) @CeO₂ catalyst recovered by filtration in Embodiment 2 is used to catalyze the selective oxidative esterification of benzyl alcohol again. The specific method is as follows.

10 mg of mesoporous Au(1%) @CeO₂ catalyst with high specific surface area and 1 mmol of benzyl alcohol were added into the reactor, and then 5 mL of anhydrous methanol was added to react for 6 hours at room temperature and 50° C. in atmospheric oxygen atmosphere. Methyl benzoate is synthesized by selective catalytic oxidative esterification, and the catalytic reaction cycle is 10 times. The analysis by gas chromatograph shows that the conversion rate of the reaction is higher than 95% and the selectivity of methyl benzoate is higher than 98%.

### Embodiment 13

Preparation of mesoporous CeO₂-loaded Au catalyst
1) Same as in Embodiment 1;
2) 500 mg of mesoporous CeO2 carrier material is dispersed in 10 mL of mixed solution of water and ethanol (the volume ratio of water to ethanol is 1:9), and 21 mL of methanol solution containing 4.75 mg/mL of HAuCl₄·4H₂O is dropped into the CeO₂ dispersion system with stirring for 8 h, then 10 mL of NaBH4 solution is dropwise added, and the stirring is further continued for 6 h. The solid product obtained by suction filtration is washed with anhydrous ethanol and deionized water respectively for three times, and then dried in vacuum at 80° C. for 3 h, thus obtaining the mesoporous CeO₂-loaded Au catalyst with high specific surface area, namely Au(2%) @CeO₂ catalyst.

The Au(2%) @CeO₂ catalyst prepared in this embodiment is used to catalyze the oxidative esterification of aromatic alcohol, and the specific method is the same as that in Embodiment 2. The results show that the conversion rate is higher than 97% and the selectivity of methyl benzoate is 95%.

### Embodiment 14

Preparation of mesoporous CeO₂-loaded Au catalyst
1) Same as in Embodiment 1;
2) 500 mg of mesoporous CeO₂ carrier material is dispersed in 10 mL of mixed solution of water and ethanol (the volume ratio of water to ethanol is 1:9), 31 mL of methanol solution containing 4.75 mg/mL of HAuCl₄·4H₂O is dropped into the CeO₂ dispersion system with stirring for 8 h, then 10 mL of NaBH₄ solution is dropwise added, and the stirring is further continued for 6 h. The solid product obtained by suction filtration is washed with anhydrous ethanol and deionized water respectively for three times, and then dried in vacuum at 80° C. for 3 h, thus obtaining the mesoporous CeO₂-loaded Au catalyst with high specific surface area, namely Au(3%) @CeO₂ catalyst.

The Au(3%) @CeO₂ catalyst prepared in this embodiment is used to catalyze the oxidative esterification of aromatic alcohol, and the specific method is the same as that in Embodiment 2. The results show that the conversion rate is higher than 98% and the selectivity of methyl benzoate is 90%.

### Comparative Embodiment 1

Same as in Embodiment 1, except that HAuCl₄·4H₂O is replaced by Co(NO₃)₂·6H₂O.

The Co(1%) @CeO₂ catalyst prepared in this comparative embodiment is used to catalyze the oxidative esterification of aromatic alcohol, and the specific method is the same as that in Embodiment 2. The results show that the conversion rate is higher than 60% and the selectivity of methyl benzoate is 76%.

### Comparative Embodiment 2

The Au(1%) @CeO₂ catalyst prepared in this comparative embodiment is used to catalyze the oxidative esterification of aromatic alcohol. The specific method is the same as in Embodiment 2, and the reaction temperature is raised to 70° C. The results show that the conversion rate is higher than 72% and the selectivity of methyl benzoate is 84%.

### Comparative Embodiment 3

Same as in Embodiment 2, the difference is that the addition amount of Au@CeO₂ catalyst is 8 mg.

The Au(1%) @CeO₂ catalyst prepared in this comparative embodiment is used to catalyze the oxidative esterification of aromatic alcohol. The results show that the conversion rate is over 90% and the selectivity of methyl benzoate is 98%.

The above describes only the preferred embodiments of this application, but the protection scope of this application is not limited to this. Any change or replacement that may be easily thought of by a person familiar with this technical field within the technical scope disclosed in this application should be included in the protection scope of this application. Therefore, the protection scope of this application should be based on the protection scope of the claims.

What is claimed is:

1. A preparation method of a mesoporous CeO₂-loaded Au catalyst, wherein the preparation method comprises using COFs-41 as a template and cerium nitrate as a cerium source, firstly loading Ce³⁺ by an impregnation method, then preparing a mesoporous CeO₂ carrier material by oxidative roasting; then, taking HAuCl₄ as a gold source and NaBH₄ as a reducing agent, loading Au nanoparticles onto the mesoporous CeO₂ carrier material by an impregnation-reduction method in an ethanol solution reaction system to obtain the mesoporous CeO₂-loaded Au catalyst.

2. The preparation method of the mesoporous CeO₂-loaded Au catalyst according to claim 1, comprising following steps: self-assembling raw materials 2,4,6-trimethylpyridine, benzoic anhydride and terephthalaldehyde at 180 degrees Celsius to synthesize a COFs-41 template material; ultrasonically dispersing an obtained COFs-41 template material in an ethanol solution containing cerium nitrate hexahydrate, followed by rotary evaporation to obtain a powder, and holding for 3 hours at 600 degrees Celsius to obtain a black solid, and roasting at 500 degrees Celsius for 3 hours to obtain a light yellow solid as the mesoporous CeO₂ carrier material;
dispersing the mesoporous CeO₂ carrier material in a mixed solution of water and ethanol, dropwise adding a methanol solution containing HAuCl₄·4H₂O while stirring, then continuously dropwise adding a NaBH₄ solution, washing and drying a product obtained by suction filtration to obtain the mesoporous CeO₂-loaded Au catalyst, the Au@CeO₂ catalyst.

3. The preparation method of the mesoporous CeO₂-loaded Au catalyst according to claim 2, wherein a mass ratio of the 2,4,6-trimethylpyridine, the benzoic anhydride and the terephthalaldehyde is 5:9:8.

4. The preparation method of the mesoporous CeO₂-loaded Au catalyst according to claim 2, wherein a mass ratio of the COFs-41 template material to the cerium nitrate hexahydrate is 1:5.

5. The preparation method of the mesoporous CeO₂-loaded Au catalyst according to claim 2, wherein a volume ratio of the water to the ethanol is 1:9, and a solid-liquid ratio of the mesoporous CeO₂ carrier material to the mixed solution is 50 milligrams: 1 milliliter.

6. The preparation method of the mesoporous CeO₂-loaded Au catalyst according to claim 2, wherein a concentration of HAuCl₄·4H₂O in the methanol solution containing the HAuCl₄·4H₂O is 4.75 milligrams per milliliter.

7. An Au@CeO₂ catalyst prepared by the preparation method according to claim 1, wherein a loading amount of Au in the Au@CeO₂ catalyst is 1-3%; a specific surface area is 153 square meter per gram and an average pore diameter is 8.7 nanometers.

8. A method for catalyzing oxidative esterification of aromatic alcohols, comprising using the aromatic alcohols as raw materials, using the Au@CeO₂ catalyst according to claim 7 as a catalyst, and anhydrous methanol as a solvent, and reacting with heating at room temperature and atmospheric pressure under an oxygen atmosphere to complete a catalytic oxidative esterification of the aromatic alcohols to synthesize aromatic esters compound.

9. The method for catalyzing the oxidative esterification of the aromatic alcohols according to claim 8, wherein a dosage ratio of the Au@CeO₂ catalyst to the aromatic alcohols is 10 milligrams: 1 millimole.

10. The method for catalyzing oxidative esterification of aromatic alcohols according to claim 9, wherein the reacting with heating is to react at 30-60 degrees Celsius for 5-48 hours.
